(19) United States
(12) Patent Application Publication
(10) Pub. No.: US 2024/0345111 A1
(43) Pub. Date: Oct. 17, 2024

(54) PIPETTE CALIBRATION MANAGING SYSTEM AND METHOD

(71) Applicant: Eppendorf SE, Hamburg (DE)

(72) Inventor: Dieter Jäger, Hamburg (DE)

(21) Appl. No.: 18/702,228

(22) PCT Filed: Oct. 21, 2022
(86) PCT No.: PCT/EP2022/079382

§ 371 Date: Apr. 17, 2024

(30) Foreign Application Priority Data
Oct. 22, 2021 (EP) 21204283.2

(51) Int. Cl. G01N 35/00

(52) U.S. Cl. CPC G01N 35/00693; G01N 35/00623; G01N 35/00732; G01N 2035/00811

(57) ABSTRACT

The invention is related to a pipette calibration managing system (PCMS), comprising a PCMS managing application to be hosted by the PCMS customer and to be run by a PCMS host server, which is accessible for clients via a data network and is configured for man-aging the access of the clients to PCMS data contained in a PCMS database stored in a PCMS data storage being located on premises of the PCMS customer, a client application to be run by a client computer and configured to communicate with the PCMS managing application via the data network, a calibration ordering software configured for providing the calibration order data to be transferred to a calibration service provider, the PCMS managing application being configured to receive calibration data from the calibration service provider via an external data network, in particular the Internet, and the PCMS data-base being configured to store the calibration data, which thereby form at least a part of the PCMS data. The invention also relates to a corresponding method.

The invention relates to a pipette calibration managing system (PCMS) and a corresponding method.

Hand-held pipetting devices are commonly used in medical, biological, biochemical, chemical and other laboratories. They are used for transport and transfer of fluid samples with small volumes, especially for precise dispensing of samples, in particular for precisely dosing and distributing liquid volumes ranging between several microliters and tens of milliliters. Two known classes of such hand-held pipetting devices differ in the respective physical principle of liquid aspiration or dispensing. Either the liquid is dispensed by the air cushion principle or the direct displacement principle is used. The first class of devices is called air cushion pipettes, the second class is called direct displacement pipettes or “dispenser”.

Air cushion pipettes use a piston-cylinder system to perform the actual measurement. An air cushion separates the sample aspirated into a plastic tip from the piston inside the pipette. When the piston slides up, a vacuum is created in the tip, which causes the liquid to rise into the tip. The air cushion moved by the piston acts like an elastic spring from which the volume of liquid in the tip hangs. In hand-held pipetting devices that operate according to the direct displacement principle, tips with an integrated piston are used as transfer vessels. This piston is coupled to the piston rod of the dispensing device during the pipetting process and takes over the actual dispensing operation. Since the piston in this case can displace the entire internal volume of the tip, essentially no air cushion is formed between the aspirated sample and the end of the piston. Both classes of hand-held pipetting devices, the air cushion pipettes and the direct displacement pipettes, are also referred to as piston stroke pipettes.

There are hand-held pipetting devices whose piston system is driven manually and those with electric drive. An electrically driven hand-held piston-stroke pipette is often controllable by at least one pipetting program in order to perform at least one type of pipetting operation in an automated or semi-automated manner. Mechanical pipettes do not use electrical energy for their operation.

Hand-held pipettes are designed for one-handed use by human users. However, there are also laboratory automats with robotic gripper arms, whose gripper tools simulate the activities of a human hand for operating a hand-held pipette and which are set up for handling and operating a hand-held pipette.

In a pipetting device, the amount of sample dispensed by a single actuation may correspond to the amount of sample aspirated into the device. However, it can also be provided that a sample quantity corresponding to several dispensing quantities is dispensed again step by step. In addition, a distinction is made between single-channel pipetting devices and multi-channel pipetting devices, wherein single-channel pipetting devices comprise only a single dispensing/receiving channel and multi-channel pipetting devices comprise a plurality of dispensing/receiving channels which, in particular, permit the parallel dispensing or receiving of a plurality of samples.

An example of a hand-held, electronic air cushion pipette is the Eppendorf Xplorer® and Xplorer® plus from Eppendorf AG, Germany, Hamburg; examples of hand-held, electronic dispensers are the Multipette® E3 and Multipette® E3x from Eppendorf AG, Germany, Hamburg.

Electric pipetting devices offer advantages over non-electric pipetting devices, as a variety of functions can be implemented in a simple manner. In particular, electric pipetting devices can simplify the execution of certain program-controlled pipetting operations by automating or partially automating them. Typical pipetting parameters for controlling such pipetting operations by means of corresponding pipetting programs concern the volume when aspirating or dispensing liquid, their sequence and repetitions, and, if applicable, their temporal parameters in the temporal distribution of these operations. Data exchange, in particular for use in the process of calibration or calibration management, with external devices can be easily implemented with electric pipetting devices.

The hand-held pipetting devices (hereinafter simply referred to as “pipettes”) described in the context of the present invention are hand-held pipetting devices of any of the aforementioned kind, which require maintenance and calibration in order to preserve the measurement and dosing accuracy over the course of time. The invention is not limited to pipettes from a single manufacturer but can be applied to pipettes from any manufacturer.

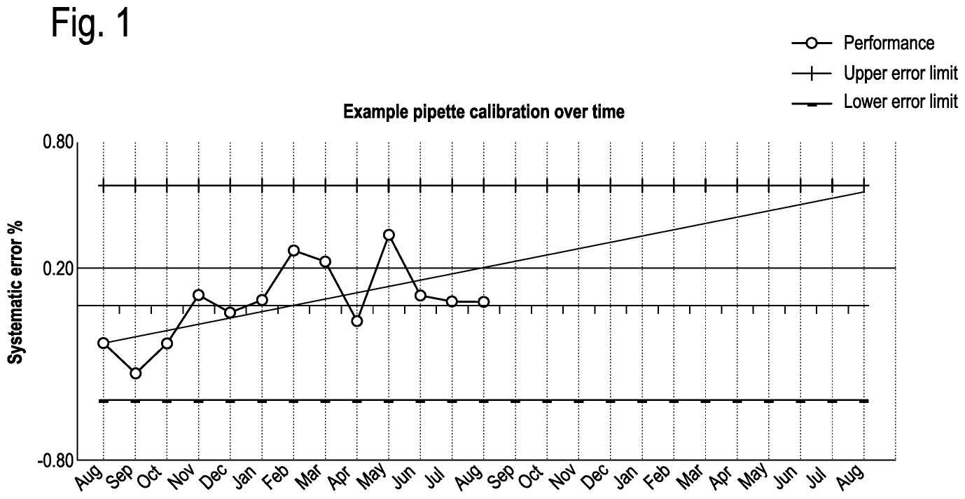

The precision and accuracy of pipettes are important for the quality of all laboratories' work results. Regular maintenance and calibration services of pipettes by a service provider assures a consistent result quality for the customer. Pipettes are precision instruments with parts subject to normal wear and tear leading to imprecision over the time. Month to month over a yearlong testing period, a pipette in standard lab use is calibrating within the error limits as shown in the graph of FIG. 1. However, wear and tear is causing this pipette's calibration to continuously just trend towards the upper limit. Without any maintenance, calibration or adjustment measures, this pipette will eventually fall out of calibration, potentially causing questionable and unreliable results.

Therefore, regular maintenance and calibration of pipettes are fundamental to their precision and accuracy. Calibration is usually applied following industrial standards. ISO 8655 provides for calibrating with three volumes to compensate for the non-linear correlation of error and volume. Within each test, the weight of a pipetted volume is determined under standard conditions. More confidence in the calibration is produced and the results become statistically more accurate when using ten measurements as recommended by ISO 8655. Calibration processes may be performed by ISO 17025 accredited service centers.

In a typical scenario of requesting the calibration of a pipette at a service provider, a user, for example an employee of a customer's company, orders a calibration service for a pipette according to the following procedure:
Ordering: a user, typically the employee of a company, has to initiate an order for requesting testing/calibration of a pipette.
Decontamination: After verifying the user's/the company's declaration of decontamination, the pipette goes through an additional decontamination process at the service provider before it is forwarded to the provider's calibration facilities.
Preventive Maintenance and Repair at the service provider: examination and calibration is performed if requested; cleaning of external and inner parts and maintenance (lubricating, exchange of wear and tear parts if necessary) of the pipette; leak testing is performed; necessary repairs may be performed.
Calibration and Adjustment at the service provider: usage of single- and multi-channel balances fullfilling ISO 8655 requirements; usage of original manufacturer pipette tips; measurement according to ISO 8655; evaluation according to manufacturer specifications (if requested to ISO 8655 or customer SOP error limits); adjustment, if device is outside of the error limits; ISO 17025 calibration certificate on request. An ISO 17025 calibration certificate may not only contain the data from the calibration protocol, but also may contain the measurement uncertainty, and the accreditation confirmation of the lab. The ISO 17025 calibration certificate with the included raw data information ensures the traceability chain for the calibration.
Labeling and Packaging: device is packed with calibration certificate and service report ready for shipment.

The average duration of pipette calibrations results in an average time of unavailability of pipettes in a customer's company, thereby reducing the productivity of a laboratory and/or the laboratory's costs. Since several individuals are involved in the above mentioned typical calibration process, significant human resources are occupied for organizing the required actions and transactions. Similarly, even obtaining information on the calibration status and availability of a respective pipette requires substantial effort. Typically, a customer's company owns a large pool of pipettes, which may also be loaner pipettes. It is the customer's desire to efficiently provide a pool of fully operational pipettes at all times to the users of the customer's company, which may require an appropriate (loaner) pipette park management. The aforementioned problems are addressed by the present invention.

The technical solution according to the invention is provided by the pipette calibration managing system (PCMS) for managing the calibration of pipettes belonging to a pipette pool of a PCMS customer, and the corresponding method, according to the independent claims. Preferred embodiments are described by the dependent claims, the description and the figures.

The pipette calibration managing system (PCMS) collates the information on the calibration status (in form of the calibration reporting data) of the pipettes belonging to the pipette pool in a central database. Thereby, the customer's company, in particular users and administrators, have access to the information as soon as the respective calibration reporting data is provided and uploaded to the database, in particular provided by a calibration service managing software, or in particular even instantly when generated by a calibration equipment and forwarded by a calibration service managing software. This allows for an accurate scheduling of laboratory work requiring operational pipettes, in particular certified pipettes. The number of pipettes in the pipette pool, which basically remain unused as a reserve, can be minimized, any superfluous purchasing of reserve pipettes can be avoided. The usage history including calibration reporting data of each individual pipette can be tracked and be documented. The time and costs required for a user to organize the calibration ordering process, the time and costs for the service provider to perform the calibration service and documenting the same, and the time and costs for the customer to obtain an overview of the pipette status in the pipette pool are significantly reduced.

Statistical data regarding the use of pipettes, calibration time, calibration details, and availability may be easily obtained from the PCMS data, and may assist the company in optimizing the pipette usage, in particular taking into account the dependence of PCMS data on any of the following factors: individual pipettes as identified by the pipette ID number, pipette types, pipette manufacturers, a pipette's age, individual users, user groups, individual laboratories or types of laboratories.

The data integrity of the calibration reporting data is ensured since the database is located on premises of the customer. Preferably, only authenticated persons (belonging to the groups including user, administrator, or provider) have access to the database, for reading or writing PCMS data, depending on the specific user group.

The pipette calibration managing system (PCMS) serves for managing the calibration of pipettes belonging to a pipette pool of a PCMS customer. The pipettes are not limited to a specific type of pipettes, or to the pipettes of a specific manufacturer. Any pipettes can be usefully tracked, which require a periodic testing and calibration. The PCMS system comprises software and/or hardware, which is, in particular, configured, in particular programmed, to fulfill the respective subfunction of the PCMS system. The PCMS may be defined to contain hardware components being connected by a data exchange network, which may be based on a LAN and/or additionally an external data network, in particular the internet, the hardware components of the PCMS system including, in particular, the PCMS host server, the PCMS data storage containing the PCMS database, the at least one client computer, the calibration ordering computer, and preferably a calibration service provider server, and preferably further components.

In the context of the present description, a software (for example: a PCMS managing application, client application, calibration ordering software, calibration service managing software, PCMS database) or a hardware (for example: a host server, a client computer, a calibration ordering computer, a calibration service provider server) being “configured” to fulfill a function generally includes the meaning that this software or hardware is programmed to fulfill the function.

The PCMS data contains calibration data from the pipettes of a pipette pool. It may also contain an As-Is protocol documenting a pipette status when the same is initially purchased or initially obtained by the customer as a loaner pipette. The frequency of calibration may vary depending on the number of users, the frequency of use of the pipettes, as well as the type of dispensed liquids and the accepted tolerances. A renewal of the conformity by ISO 8655 may be recommended at least once per year. The calibration/renewal of conformity may consist of a pipette maintenance and an As-Left calibration. These may adhere to ISO 8655 criteria, ensuring that the pipette meets conformity to ISO 8655 requirements or even tighter specifications like manufacturer specifications. The As-Is protocol is performed before the As-Left calibration or renewal of conformity including maintenance. This protocol shows the calibration status of the pipette as received from the user, and is indicative of the pipette's performance prior to the renewal of conformity. The As-Is protocol may be additionally contained in the calibration recording data. An ISO 17025 calibration certificate not only contains the data from the calibration protocol, but also contains the measurement uncertainty, and the accreditation confirmation of the lab. The ISO 17025 calibration certificate with the included raw data information ensures the traceability chain for the calibration.

The PCMS data contains calibration data from the pipettes of a pipette pool, in particular including at least one timestamp showing the time when the calibration and/or a calibration certificate was finished and/or uploaded to the PCMS database and/or an information on the completed calibration and/or the calibration certificate was sent to the customer and/or the comissioning user.

The PCMS data may contain a collection of the calibration data of a specific pipette as identified by the respective pipette ID number, over the time. Such a pipette calibration history allows to derive information on the specific quality of the pipette, and/or the intensity of use of the specific pipette. The PCMS data may contain a collection of the calibration data of a specific user as identified by the respective user ID number, over the time. Such a user history allows to derive information on the activity of the user.

The PCMS managing application is hosted by the PCMS customer and run by a PCMS host server, which is accessible for clients via a data network and is configured for managing the access of the clients to PCMS data contained in a PCMS database (within this specification also referred to as “database) stored in a PCMS data storage being located on premises of the PCMS customer. The PCMS managing application is preferably hosted on a Server running on Windows, or any other operating system, for example Linux or Unix. The PCMS managing application is preferably configured, in particular programmed, for user registration. A registered user will have access to the database, in particular to entries in the database being correlated with the user ID number of said user. The PCMS managing application is preferably configured, in particular programmed, for dispatching of automated messages, preferably E-mail messages, to the user, in particular to the commissioning user, whose calibration order is completed by the upload of the calibration reporting data to the PCMS database. Preferably, the PCMS managing application comprises, or is, an application programming interface (API). An API is known to be a connection between computers or between computer programs. It is a type of software interface, offering a service to other pieces of software. A document or standard that describes how to build or use such a connection or interface is called an API specification. A computer system that meets this standard is said to implement or expose an API. The host server preferably implements the API, which represents at least a part of the PCMS managing application.

The PCMS managing application is configured to receive calibration data from the calibration service provider via an external data network, in particular the Internet. The PCMS managing application is, preferably, configured to allow the provider uploading the calibration to the PCMS database, in particular via the calibration service managing software. The PCMS managing application is, preferably, configured to download the calibration data into the PCMS database from a data storage provided by the provider. The PCMS database is configured to store the calibration data, which thereby form at least a part of the PCMS data.

The calibration data contain
the pipette ID number of said pipette
and/or the calibration order ID number,
and calibration reporting data, which contain calibration status data and/or a calibration certificate.

The calibration status data contain the information if a calibration of said pipette was successfully performed by the calibration service provider.

The calibration certificate contains the information certifying the authenticity of a successful calibration of the pipette.

Preferably, the host server is located on premises of the PCMS customer (also referred to as “customer”). Preferably, the host server may be a single server or a server cluster. Preferably, the host server runs the PCMS managing application and/or stores the PCMS database. Preferably, the host server is accessible for the clients via a local area network (LAN) on premises of the customer, or may be accessible via the Internet, in particular using an authentication process for the user authentication. Preferably, the host server is not located on premises of the PCMS service provider but is located remote thereof. The host server may be a virtual machine, in particular a system virtual machine, as provided by the computer systems of the customer.

The PCMS data storage may be part of the host server. The PCMS data storage may be a data storage device, which can be accessed by the host server and the PCMS managing application, or by the calibration service managing software, via a data connection, in particular via a LAN (or also WLAN) of the customer or via an external data network, in particular the Internet. The PCMS data storage preferably contains a non-volatile storage medium being a type of computer memory that can retain stored information even after power is removed, in particular one or more hard disk drives, solid state disks, optical discs and/or magnetic tape.

Preferably, the PCMS database is selected from at least one of the following types: a relational database, a key/value database, and a modified relational database. Further, in some embodiments the data entries in the database are related hierarchically. The PCMS database contains and/or represents data objects and, possibly, metadata relating thereto. Preferably, the PCMS database is hosted on a server running on Windows. The PCMS database may be a SQL database.

The client application is run by a client computer and configured to communicate with the PCMS managing application via the data network. The client application is preferably configured to communicate over the data network, in particular a LAN, with an API, being a part of the PCMS managing application or forming the same, and/or to directly exchange data with the PCMS database-directly meaning without having the exchanged data being processed by the PCMS managing application.

The client application is configured to perform the following steps:
access the PCMS database,
receive the calibration reporting data in dependence on the pipette ID number and/or the calibration order ID number,
optionally: output the calibration reporting data to the user.

The client application is preferably configured to receive the calibration reporting data in dependence on user ID number.

The client application, preferably, is configured to display a graphical user interface (GUI). The GUI may contain at least one output screen with information in a display of the client computer. One output screen may be referred to as “my pipettes screen” and may show one or more or all of the following information about currently and/or past owned pipettes:
pipette status: [for example: in use, in service (calibration service), out of order]
pipette ID number
description (for example: Manufacturer, Type, Volume range)
last calibration date
next calibration date
currently active calibration order ID number (only exists if the pipette is in service)

One output screen may be referred to as “order screen” and may show one or more or all of the following information about currently and/or past owned pipettes:
all available pipette information (manufacturer, type, volume range, channels, au-toclavable)
all past service records (status, service level, certificate, date of service, calibration order ID number)
all changes in ownership

One output screen may be referred to as “loaner screen”. In the loaner screen all available loaner pipettes may be listed. It may be configured to be filtered by manufacturer, type, number of channels and volume range. A loaner pipette may be added to a cart. The reservation may be completed in a cart tab. A “my reservations” tab may show the status and pickup date of all past and current reservations. The pipettes may then be picked up at a shop, which may be a room on premises of the customer.

One output screen may be referred to as “search screen”. The search screen may be configured to allow searching for any pipette, calibration service record or calibration order in the database.

The client computer may be any processing device, capable of running the client software and capable to connect to the Internet. Any processing device may be utilized, including for instance, a personal computer, a laptop, a tablet computer or a smart phone. The client computer is, preferably, part of a local area network of the customer, wherein the client computer preferably is connected to the host server via the LAN. The client computer, in particular, has a display for outputting a GUI allowing the interaction of a user with the PCMS, in particular the client application.

The term client is understood here as for describing a client computer, a client software or client application, respectively, the user using the client application, depending on the context.

Preferably, the client application and/or the PCMS managing application are configured for a limited access, which means that access is allowed for an authenticated user and is prohibited for a non-authenticated user. The access is preferably restricted to the company that hosts the database (the customer) and the provider. The access is preferably restricted such that only employees of the respective company and the provider will have access to the functions of the client application. Preferably, the client application and/or the PCMS managing application are configured to register a user, before the user is allowed to use the functions of the client application and/or the PCMS managing application. A user may then be required to choose a valid login and password, for implementing a user authentification process.

Preferably, the client application and/or the PCMS managing application are configured for letting the data exchange with the PCMS database be secured by appropriate means (e.g. VPN, tunneling, mulesoft or others). Preferably, the client application and/or the PCMS managing application are configured for letting the provider access the company network to upload certificates and manage App-Users.

The company of the customer may use company badges for identifying each user (hereinafter “user badges”), which may contain an RFID chip or any other machine readable information carrier. In case that a reading device, for example an RFID reader, is connected to the client computer and/or a calibration ordering computer and/or the host server, an authentication of the user may be implemented by the client application and/or the calibration ordering software and/or a PCMS managing application by evaluating the result of a process of reading the user badge and then comparing the user data contained in the user badge with a list of registered users, which may be hosted by the host server.

Different user classes used with different permissions for the database may be provided.

An App-User may be provided, which may be a customer's employee logged in over the client application. A Shop-User may be provided, which may be the default user when logging in over the calibration ordering software. A user badge may be required to login as a

Shop-User-alternatively an App-User password can be used. A Provider user may be provided, which may be an employee of the provider's company either stationed at the calibration ordering computer on premises of the customer or stationed for using the calibration service managing software, in particular on premises of the provider. An Admin user may be provided, which may be a super user used for database maintenance and debugging.

There are substantially three separate applications for App-User, Shop-User and Provider user. The Admin user may use a third-party SQL client to manage the database. A list of access rights of the user classes may be hosted by the host server. The list of access rights of the user classes may be implemented according to one or more or all relations listed in the following table:

| | App- | Shop- | | |
|---|---|---|---|---|
| | User | User | Provider | Admin |
| Register new App-User | x | | | x |
| View pipette status and information | x | | x | x |
| (any) | | | | |
| View order status and information | x | | x | x |
| (any) | | | | |
| View certificate information and | x | | | x |
| request certificate (sent per mail) | | | | |
| Create loaner reservation | x | x | x | x |
| Complete loaner reservation (Hand | | x | | x |
| out reserved loaner pipettes) | | | | |
| Edit user settings (for current user) | x | | | x |
| Edit user settings (for other users) | | | | x |
| Register user badge for user | | x | | x |
| Create service order for owned | | x | | x |
| pipettes | | | | |
| Create service order for loaner | | | x | x |
| pipettes | | | | |
| Change pipette owner | | | x | x |
| Upload certificate | | | x | x |
| Add and edit pipette type | | | x | x |
| Add and edit pipette | | | x | x |
| Export database (for statistics) | | | x | x |

The calibration ordering software is configured for
receiving a pipette ID number identifying a pipette of the pipette pool,
optionally: receiving a user ID number identifying a commissioning user,
generating calibration order data containing a calibration order ID number and said pipette ID number,
providing the calibration order data to be transferred to a calibration service provider.

Preferably, the calibration ordering software is configured for one or more or all of the following functions:
create a user by connecting to the PCMS managing application and running a process of user registration at the PCMS managing application
connecting the user badge to a user account, which is created upon registration of the user at the PCMS managing application, for validating the user ID number and the user badge
create an asset, meaning creating “the pipette” by entering the pipette ID number into the system, a process which may executed by reading an information carrier being attached to a specific pipette, wherein the information carrier may be a bar code, in particular a 2D bar code, e.g. QR code, an RFID chip or any other information carrying medium; the reading requires a suitable reading device, e.g. an 2D-bar code reader, being connected to the calibration ordering computer
make a service order for my pipettes
running a digital signature process for a decontamination confirmation
reserve loaner pipettes
record information regarding the pickup of reserved loaner pipettes

A decontamination confirmation is the information of the commissioning user (the registered user who initiates a calibration service order), that the pipette was professionally decontaminated, e.g. by using an autoclave running a suitable autoclave process. The decontamination may depend on the safety level of the laboratory where the pipette was used or the type of contaminants which were pipetted by the pipette. A digital decontamination confirmation may be implemented by a cross check on a GUI to be set by the undersigning user, who is identified by his valid login and/or his user badge.

In the context of the present description, an ID number (for example: user ID number, pipette ID number, calibration order ID number) is understood to be data, which are suitable to unambiguously identify an item within a plurality of items, for example to identify an individual user within a group of registered users, each having its own individual user ID number. The term ID number is not restricted to mean a sequence of numerals or digits, but may refer to any sequence of characters, or to any information, which is suitable to guarantee the required distinguishability.

The calibration ordering computer may be any computer capable for implementing the required functions, in particular a computer having a display for using a GUI for the user interaction. The computer may be, for example, a tablet PC, a PC, a mobile phone.

The calibration ordering computer may be connected with a reading device for reading an identification document of a user, for example a user badge. Such a reading device may be, depending on the information carrier used in the identification document, an RFID reader, or a barcode reader. Moreover, a reader for biometric authentication may be provided, wherein a biometrical identification information may be compared with a table containing all biometrical identification information, which table unambiguously correlates a specific biometrical identification information with an individual user, who was registered by storing his biometrical identification information in combination with his name, for example. Such a table may be accessible, preferably solely accessible, for the PCMS managing application, which in turn, may be accessed by the calibration ordering software or other software.

The calibration ordering computer may be connected with a reading device for reading an information carrier attached to a pipette, the information carrier containing the pipette ID number, and, for example, additional information, for example, the pipette manufacturer, a pipette model/type description, a date of manufacturing, date of last calibration etc.

Preferably, the pipette managing system comprises a calibration service managing software hosted by the calibration service provider server and configured to fulfill one or more or all of the following functions:
receive the calibration order data from the calibration ordering computer via a data network, in particular the Internet,
output the calibration order data, for initiating the calibration of said pipette to be performed manually or automatically by the calibration service provider, wherein said outputting may include forwarding at least a part of the calibration order data to a computer device, which may be located on premises of the provider.
providing the calibration data, including, preferably an automated transfer of calibration data from one or more calibration equipment, or from a calibration software of such a calibration equipment, to the calibration service managing software.
transferring the calibration data to the PMS database, preferably by uploading the calibration data to the database, via the data network, in particular the Internet.

Said computer device may be a calibration equipment, which is configured to at least contribute in performing the processes required for calibration, which includes a testing procedure evaluating if the pipette is operating within predefined range of error tolerances around the ideal values, and, if the pipette's condition is found to be outside the tolerated borders, to adjust at least one correction factor, which returns the pipette's operation back to the tolerated area around the ideal value(s). Said computer device may also be a PC or other computer, which is connected for data exchange with the calibration equipment. The calibration equipment is described below in more detail.

The calibration service managing software is preferably configured to fulfill one or more or all of the following functions:
Update a pipette information/status regarding a specific pipette ID number: in particular by receiving data from a computer device, which is a part of the calibration equipment or forms the same, and by deriving the pipette information/status of a pipette having said pipette ID number from such data; and/or by uploading the pipette information/status as a part of the calibration data of said pipette ID number to the database.
Change pipette owner; the owner may initially be the commissioning user, by default.
Update order status, for example by setting a parameter indicating the order status to be one of a group of possible order status, including “order is open and being executed”, “order is fully processed and complete”, “order is cancelled”, “order was amended” etc.
Update service records, including storing information from a calibration order, in particular calibration data
Make service order, in particular for loaner pipettes
Upload one or more calibration certificate, in particular having a PDF file format, to the database.
Export database statistics report

The calibration service managing software may be a software running on Microsoft Windows. The PCMS may comprise a calibration service provider server being located on premises of the calibration service provider, said calibration service provider server preferably running Microsoft Windows operating system, and/or said calibration service provider server preferably being configured to run the calibration service managing software. Said calibration service provider server is preferably connected to a data network, in particular the Internet, which allows to exchange data with the host server, and/or the database, and/or the PCMS managing application.

The pipette calibration managing system according to the invention may include at least one calibration equipment and/or at least one computer device configured to exchange data with the at least one calibration equipment and/or control the at least one calibration equipment. Said computer device may be a calibration equipment, which is configured to at least contribute in performing the processes required for calibration, which includes a testing procedure evaluating if the pipette is operating within predefined range of error tolerances around the ideal values, and, if the pipette's condition is found to be outside the tolerated borders, to adjust at least one correction factor, which returns the pipette's operation back to the tolerated area around the ideal value(s). Said computer device may also be a PC or other computer, which is connected for data exchange with the calibration equipment. The calibration equipment may include a precision balance suitable to measure the weight of volumes, which are relevant for the respective pipette to be calibrated, said range being for example taken form the range between 0.5 μl and 10 ml, for example. The calibration equipment may include a robotic device configured to automatically handle the pipette, including a robotic gripper for holding and operating the pipette, thereby mimicking the behavior of an ideal expert user executing the pipetting process with repeatable precision. The calibration equipment may include a climate control chamber (stability chamber), which is capable to accommodate the precision weight and/or the pipette and/or the robotic device in a chamber, which is conditioned to standardized climate values, regarding a standardized temperature, and/or a standardized humidity, and/or a standardized gaseous composition of the surrounding air.

The PCMS is preferably configured to comprise the at least one calibration equipment being connected via a data connection to the calibration service provider server, which is connected to the PCMS database storage via a data network, in particular the Internet. This way, the PCMS is capable to transfer calibration data immediately to the PCMS database, once they are generated by the at least one calibration equipment, via the calibration service provider server, in particular without requiring the interaction of a user with the PCMS system. Even the calibration certificates may be automatically generated and immediately transferred to the PCMS database.

Preferably, the at least one calibration equipment, in particular a calibration software running on the calibration equipment, is configured and respectively, programmed, to exchange data, in particular calibration data, with the PCMS, in particular with the PCMS managing application and/or the PCMS database. Preferably, the at least one calibration equipment, in particular a calibration software running on the calibration equipment, is configured and respectively, programmed such that an upload of said data, in particular said calibration data, is preferably automatically and substantially immediately initiated or initiated within a predetermined after a calibration of a pipette is finished, said upload transferring said data (calibration data) from the at least one calibration equipment, or from a computer device forming part of the at least one calibration equipment and/or being connected to the same, to the PCMS host server and/or the PCMS database.

The pipetting devices are, preferably, connectable or connected to the PCMS host server or to a computer being connected to the PCMS host server, by a wireless data connection, in particular via a Wireless Local Area Network (WLAN), Blutooth or WiMAX.

The pipetting device may be programmed such that a completion message data is sent, from the pipetting device or a computer device connected to the pipetting device, to the PCMS managing application or respectively, to the PCMS host server, once the pipetting device is the first time connected to the PCMS managing application or respectively, to the PCMS host server (directly of via another computer device being connected to the PCMS host server), after the completion of a calibration of said pipetting device. This way, the PCMS managing application or a computer device being connected/connectable to the at least one pipetting device is programmed such that said pipetting device is registered as an active (active: returned or introduced to the pool of pipettes) and properly working (properly working: validly calibrated, in particular dertified by the certificate) pipetting device of the pipette pool, and this status data may be stored in the PCMS data base as a status information of said pipetting device. The PCMS database may contain the status data of one or more or all pipetting devices of the pipetting pool. The pipetting pool may comprise all the pipettes from a company, or from a subgroup of the company, for example from a department, a laboratory, or a working group or from an individual user.

The client computer and/or a pipetting device of the pipette pool may contain an indication device, which indicates the status of the calibration of the pipetting device, in particular depending on the pipette ID number. This may be achieved by a suitable programming of the client application and/or the control software controlling the pipetting device. The status (calibration status) may be “calibrated”, “calibration needed”, “a certificated calibration was verified”, “decommissioned device”, for example. The indication device may be a display of the pipette, which may show an indicator, in particular a color mark, a symbol or a text, whose visual appearance and/or content depends on the status data of the pipetting device. The indication device may also be an LED or another visual indicator, whose appearance depends on the status data of the pipetting device: for example, blue color indicating the status “a certificated calibration was verified”, green color indicating “calibrated” (calibrated meaning perferabyl: calibrated by the calibration equipment, but not yet using the status data from the PCMS server), “orange color: calibration needed”, “red color” meaning “decommissioned device or repair needed”.

The PCMS managing application may be programmed to transfer the status data of a pipetting device to the pipetting device via a (wireless or wired) data connection once the pipetting device is connected to the PCMS host server via said data connection, in dependence of the pipette ID number. Hereby, the status data were provided by the PCMS host server as extracted from the calibration reporting data/calibration data, which were received from the calibration service provider/from the calibration equipment or a computer device connected to the calibration equipment, via an external data network (6), in particular the Internet. This means, the information on the status of a pipetting device is guaranteed to be taken from the verified data belonging to the sphere of the customer (the PCMS database), and it can be guaranteed that the status information shown on the pipetting device is always a status information which is being documented, by storage in the PCMS database. In case that the PCMS database also stores the calibration certificates, the status data is a “certificated status data”. In contrast, when the pipetting device is returned from the calibration service provider and, in particular, not yet connected to the PCMS host server, the status may be “calibrated” but not certified and documented by the PCMS database. For example, a pipetting device which is not owned by the customer, which means that the respective pipette ID number is not registered by the PCMS managing application, may show a “calibrated” indicator but will not show a “a certificated calibration was verified” indicator, even if connected to the PCMS host server. In case that a registered pipetting device of the pipette pool was successfully calibrated by the calibration service provider and was connected to the PCMS host server, which-however-did not (yet) correctly receive the calibration data for the pipetting device from the calibration service provider, the indication device of said pipetting device may show a “calibrated” indicator but will not show a “a certificated calibration was verified” indicator, even if connected to the PCMS host server.

The pipetting device, or respectively the control software of the pipetting device controlling the function of at least the status indicator, is, preferably, programmed such that the status indicator is set in dependence on the status data as received by the pipetting device (or respectively, a computer device controlling at least the indication device) from the PCMS managing application (PCMS host server), via a wireless or wired data connection, subsequent to a calibration of said pipetting device, said status data forming part of the calibration reporting data received by the PCMS managing application (PCMS host server) from the calibration equipment (or a computer device connected to the calibration equipment). As an alternative, the pipetting device, or respectively the control software of the pipetting device controlling the function of at least the status indicator, is, preferably, programmed such that the status indicator is set in dependence on the status data as directly received by the pipetting device from the calibration equipment (or a computer device connected to the calibration equipment)-“directly” meaning: without utilizing the status data as taken from the calibration reporting data, which are transferred to the PCMS host server after the calibration. An advantage of using the status data as received from the PCMS host server (and not directly from the calibration equipment) is that the user of the pipetting device can be sure that the pipette was calibrated under the supervision of the company, which guarantees a certificated pipetting device, because the calibration reporting data, preferably including the calibration certificate, is stored in the PCMS database and therefore the history and quality of the pipetting device is well documented.

programmed such that the status indicator is set in dependence on a data exchange of the pipetting device and the PCMS managing application (PCMS host server) subsequent to a calibration of said pipetting device, which data exchange verifies that the calibration reporting data were transferred from the calibration equipment (or a computer device connected to the calibration equipment) to the pipetting device (meaning a computer device being part of the pipetting device) a successful calibration

After the calibration of the pipetting device, the calibration data, which is reveived by the PCMS managing application and/or the PCMS database, is stored in the PCMS database and the PCMS managing application is programmed such that said pipetting device is registered as a properly working (properly working: validly calibrated, in particular dertified by the certificate) pipetting device of the pipette pool, and this status data may be stored in the PCMS data base as a status information of said pipetting device.

The PCMS host server or at least one computer connected to the PCMS host server may be programmed to output (via a display, for example) information on the status data (also referred to as calibration status data) of a pipetting device, in particular once the status of the pipetting device has changed from “currently in a calibration process” to “successfully/not successfully calibrated” or from “not working properly” to “working properly”. The status “currently in a calibration process” may be automatically assigned to the pipetting device or respectively, the pipette ID number, as a status data, once a calibration order is initiated by the calibration ordering software, after generation of the calibration order data and/or the calibration order ID number. The calibration reporting data may also be output, together with the calibration data.

The calibration reporting data preferably includes at least one, several or all of the following data: the pipette ID number, a correction factor, which was used to calibrate the pipetting device; measurement data and preferably, a corresponding measurement uncertainty; a calibration certificate, in particular a ISO 17025 calibration certificate of the laboratory which performed the calibration and/or an DIN EN ISO 8655 certificate of the calibration process; the measurement results required for the calibration.

The measurement of the calibration typically includes pipetting (aspirating into a pipette tip using the pipetting device and discharging the liquid from the pipetting device) a predefined volume (set volume, e.g. 10 μl) of a defined liquid (typically: degassed and destilled water or deionized water, having a verified temperature), under defined climatic conditions, thereby obtaining a liquid sample, measuring the mass of the liquid sample using a calibrated balance, calculating the volume of said liquid sample from the mass using the calculation factors of the defined climatic conditions (temperature, air humidity) and comparing the measured volume volume of said liquid sample with the set volume. Typically, multiple (number N, e.g. N=10) such measurements are performed, and an average of the measured volumes is determined and is compared with the set volume, preferably taking into account the systematic error and/or the random error. The measurement series may be obtained for different set volumes, for example at 10%, 50% and 100% of the nominal volume of the pipette. A calibration factor, or respectively, correction factor, which is internally used by the pipette to control the movement of the piston of a pipetting device in the correct way, may be, or may be a mathematical expression depending on, the quotient {measured (averaged) volume}/{set volume}.

Preferably, the PCMS managing application or a computer device connected/connectable to the at least one pipetting device is programmed to transfer supervisor data to at least one pipetting device of the pipette pool, in particular in dependence of the pipette ID number, automatically or following the manual input of a supervisor user at the PCMS host server or said computer device. The pipetting device may be programmed such that a visual and/or acoustical information is output by an information device forming part of the pipetting device or a computer connected to the same, in dependence on the supervisor data received from the PCMS host server or said computer device. The pipetting device may be programmed to block one or more or all electronically controllable functions of the pipetting device, in dependence on the supervisor data. For example, the pipetting device may be programmed to be decommissioned by the supervisor data and/or programmed such that a visual and/or acoustical information is output by an information device forming part of the pipetting device or a computer connected to the same. The information device may be a display or light source or speaker. The PCMS host server or said computer device may be programmed to evaluate-automatically or after receipt of calibration data following a calibration process-, in dependence of the pipette ID number, the calibration data of a pipetting device, in particular s series of historical calibration data, in particular for detecting that a calibration factor, in particular the correction factor, exceeded a predetermined limit. In case that a pipetting device was correctly calibrated by the calibration equipment, it is still possible that a supervisor or supervising software decides that a pipetting device is not working reliably enough. In this case a repair or decommission of the pipetting device may be useful.

On-premises software is installed and runs on computers on the premises of the person or organization using the software (the customer), rather than at a remote facility such as a server farm or cloud. The PCMS managing application preferably is an on-premises software being installed and running on the PCMS host server on the premises of the person or organization using the PCMS managing application. On the other hand, the calibration software, in particular the calibration service managing software, preferably is being installed and running on a calibration service provider server on the premises of the person or organization providing the calibration service. Therefore, the calibration software may be considered being an off-premises software for the customer. The PCMS managing application may be considered being an off-premises software for the provider. The at least one calibration equipment may be configured to generate. and provide calibration data of one or more pipettes. The at least one calibration equipment may be configured to generate. and provide raw data of at least one measurement required for testing and/or calibrating the pipette, as a part of the calibration data—an ISO 17025 calibration certificate with the included raw data information ensures the traceability chain for the calibration. The calibration certificate preferably is an ISO 17025 calibration certificate, wherein the underlying calibration was preferably done following ISO 8655.

The calibration equipment is preferably configured to transfer the calibration data of a pipette to an information carrier, which is attached or is to be attached to the pipette. The information carrier may comprise a data storage, in particular an RFID chip, or a bar code, in particular a 2D-barcode, e.g. a QR code. The information contained in the information carrier may be readable and understandable for a human user, which is the case for a clear text containing a value and its description, for example the date of calibration and the description “date of last calibration”. The information contained in the information carrier preferably is purely machine-readable, i.e. not readable and understandable for a human user, which is the case for an RFID chip, or a bar code, in particular a 2D-barcode, e.g. a QR code, for example. A clear text may be machine-readable by using a camera and implementing image recognition.

The PCMS, in particular the client computer, the calibration ordering computer, and/or the calibration service provider server, and/or the calibration equipment, preferably contains a reader for reading the information from an information carrier, which is in particular attached to the pipette, in particular using the software of the respective hardware, which is then programmed to implement such a reading process. The attachment may be a sticker disposed on the outer surface of the pipette, or a data storage attached to the casing or any other part of the pipette.

Preferably, the PCMS includes at least one information carrier, which carries information regarding the calibration of a pipette. The information may be similar or equal to information contained in the calibration data. The information may include any of: the pipette ID number, a user owning the pipette, the user who commissioned the recent calibration, the next (future) calibration date, the manufacturer of the pipette, the pipette type/model, and/or the calibration order ID number.

Preferably, the PCMS includes at least one pipette comprising said information carrier.

The renewal period of a pipette, which is understood to be the period between the date of a previously completed calibration and the date of a scheduled subsequent calibration of a pipette, may be predetermined or be set by a user. A typical renewal period is one year, but may be adapted to the needs of a specific pipette, pipette type, or pipette application type.

The calibration service managing software and/or the PCMS managing application and/or the client application may be configured to set the renewal period of a pipette, or generally of all pipettes. The calibration service managing software and/or the PCMS managing application and/or the client application may be configured to output the renewal period of a pipette, or generally of all pipettes, to a user. The calibration service managing software and/or the PCMS managing application and/or the client application may be configured to output the renewal period of a pipette, or generally of all pipettes, in a display. The calibration service managing software and/or the PCMS managing application and/or the client application may be configured to output the renewal period of a pipette, or generally of all pipettes, in an output screen of a GUI used for the respective software.

The calibration service managing software and/or the PCMS managing application and/or the client application may be configured to output in a display, or respectively in an output screen of a GUI, one or more or all of the following information:
an information to the user about the renewal period of a pipette, or generally of all pipettes
the ending date of the renewal period of a pipette, or generally of all pipettes, in particular regarding a specific user ID number
the date of the most recent calibration of a pipette, or generally of all pipettes, in particular regarding a specific user ID number, in particular more or all dated of calibration of said pipette

Preferably, the PCMS, in particular one of its software components, in particular the calibration service managing software and/or the PCMS managing application and/or the client application may be configured to evaluate and/or output to the user the renewal status of a pipette, which means that it is evaluated (and output, if needed) if a pipette, or respectively each pipette, at the current day of the evaluation,
first case (1; “normal phase”) lies within the renewal period of the respective pipette, and/or
second case (2; “reminder phase”) if the pipette, or respectively each pipette, is currently situated within a reminding period within the renewal period of the pipette, the reminding period ending with the end date of the renewal period. The reminding period may be set by a user, and may be 4 weeks by default, and/or.
third case (3; “overdue phase”) is situated after the renewal period of the pipette.

Preferably, the PCMS, in particular one of its software components, in particular the calibration service managing software and/or the PCMS managing application and/or the client application may be configured to graphically indicate the renewal status of one or more or all pipettes, in particular regarding a specific user ID number, to a user by showing a graphical representation, in particular a graphical symbol, graphical marking, or color marking, on a display, or respectively on the output screen of a GUI, in particular next to a pipette ID number of the respective pipette, such that the user can unambiguously correlate the graphical representation of the renewal status to the pipette ID number. Said graphical representation may implement a color coding for the renewal status, for example following the logic of traffic lights, wherein a first color, e.g. “green”, refers to the “normal phase”, a second color, e.g. “yellow”, refers to the reminder phase, and a third color, e.g. “red”, refers to the overdue phase of the renewal status. Using such a graphical representation, in particular a color coding, the PCMS uses a highly ergonomic solution for allowing the many users of a PCMS to efficiently recognize and decide, which pipette has to be send to calibration, in order to guarantee a permanent availability of fully operational and calibrated pipettes. It is plausible that using such an ergonomic system avoids tiring of the users and rather motivates the user to evaluate the renewal status for deciding to send one or more pipettes to the calibration service. Over the time, such a system will improve the efficiency of the laboratory work performed with such a fully calibrated pipette pool.

The invention is also related to a pipette calibration managing method (PCMM) for managing the calibration of pipettes belonging to a pipette pool of a PCMS customer, the method using, in particular, the pipette calibration managing system according to the invention and comprising the steps:
optionally: receiving a user ID number of a user, who belongs to the PMS customer, using the calibration ordering software,
receiving a pipette ID number identifying a pipette of the pipette pool, using the calibration ordering software,
generating calibration order data using the calibration ordering software,
providing the calibration order data to be transferred to a calibration service provider, in particular via the Internet,
receive and store calibration data from the calibration service provider via an external data network, in particular the Internet, in the PCMS database, the calibration data thereby forming at least a part of the PCMS data,
optionally: let a user, via the client application:
   access the PMS database,
   receive the calibration reporting data in dependence on the pipette ID number and/or the calibration order ID number and/or the user ID number
   optionally: output the calibration reporting data to the user.

The system and the method according to the invention are applicable, for example, in research or industry related to pharmacy, biomedical sciences, biotechnology, in particular plant biotechnology, microbiology, chemistry or nanotechnology.

Further preferred configurations of the system according to the invention and the method according to the invention emerge from the following description of the exemplary embodiments in conjunction with the figures and the description thereof. If nothing else is described or if nothing else emerges from the context, the same components of the exemplary embodiments are substantially characterized by the same reference signs. In detail:

FIG. 1 contains a chart showing the measurement of accuracy for a specific pipette, the measurement being repeated over time.

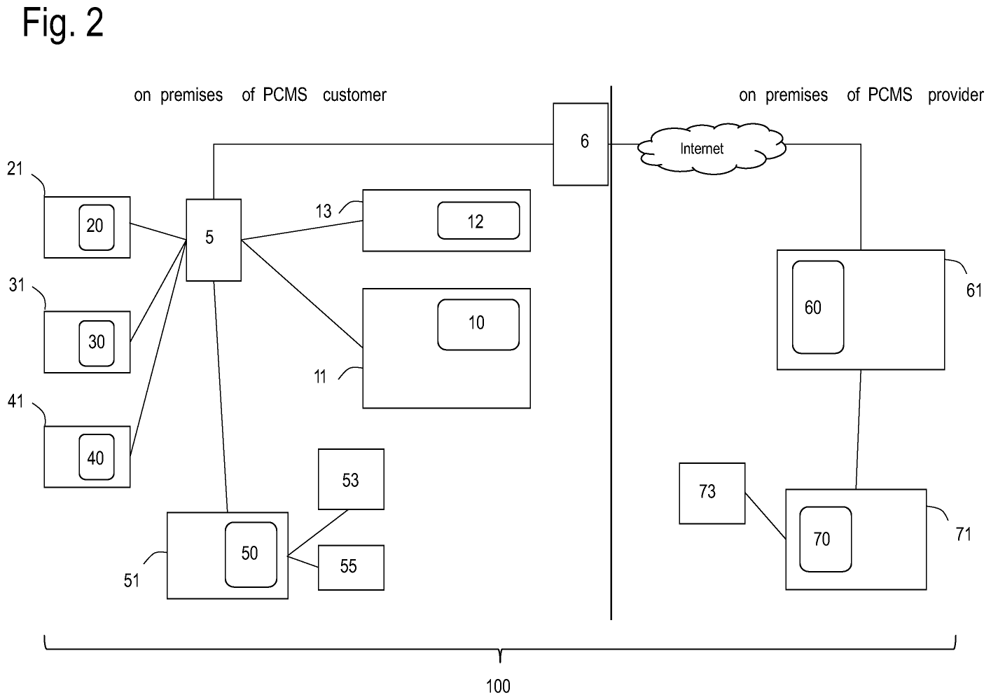

FIG. 2 shows a pipette calibration managing system according to an example of the invention, and its components.

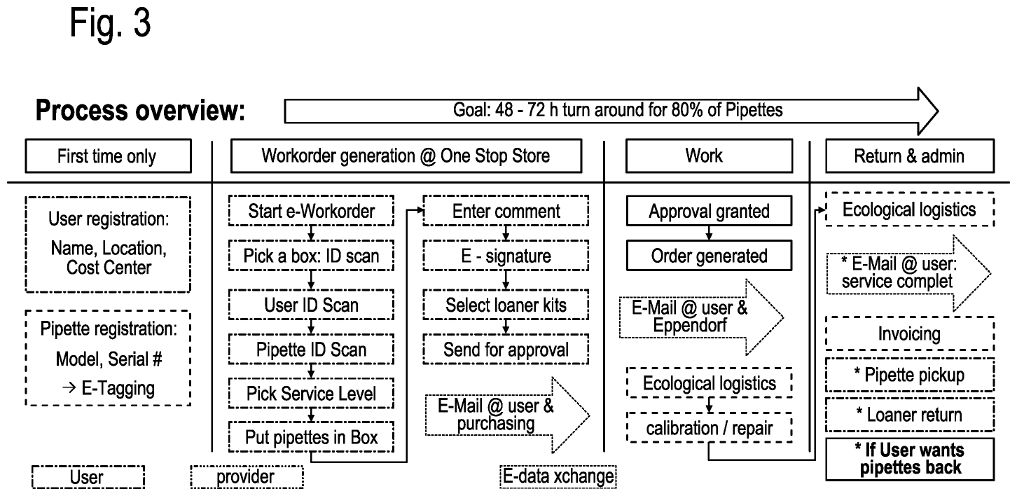

FIG. 3 schematically shows a process overview of the calibration process as managed by the pipette calibration managing system shown in FIG. 2.

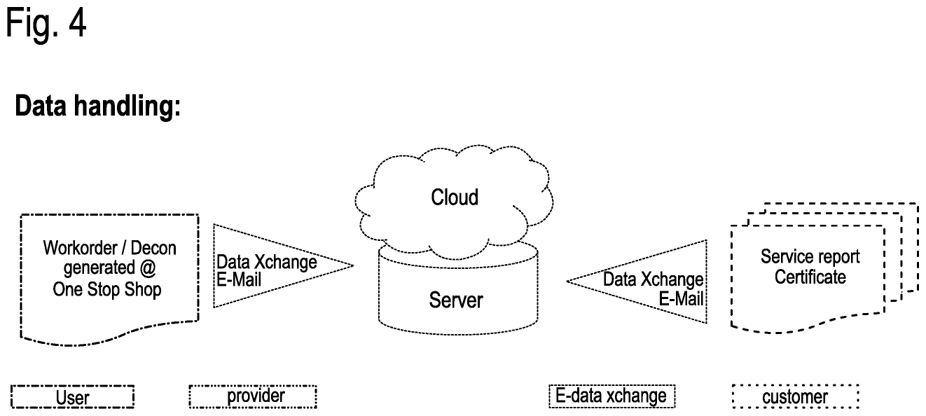

FIG. 4 schematically shows the data handling of the pipette calibration managing system shown in FIG. 2.

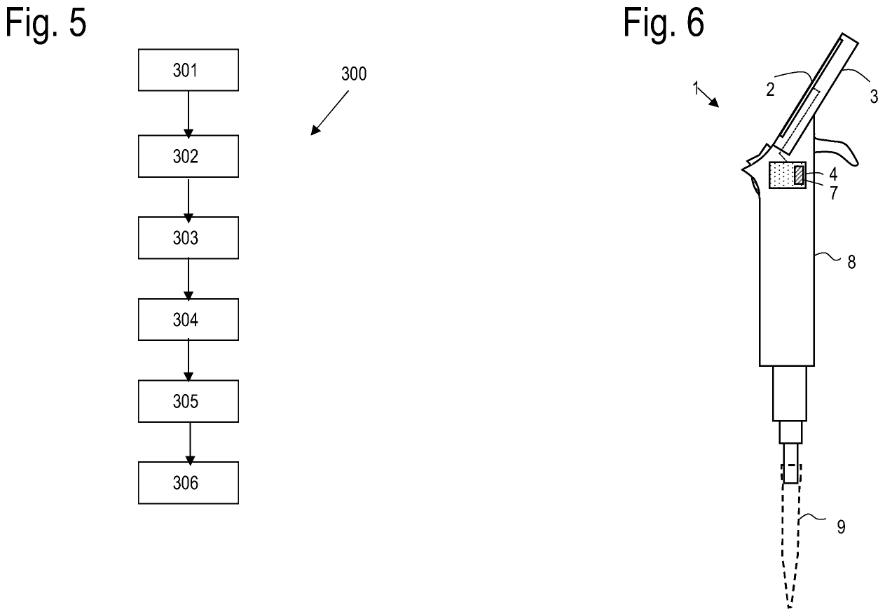

FIG. 5 schematically shows the pipette calibration managing method (PCMM) according to the invention, according to a preferred embodiment.

FIG. 6 shows an embodiment of a pipette having an information carrier being part of the system of FIG. 2, in a preferred embodiment of the invention.

FIG. 1 shows a chart showing the measurement of accuracy for a specific pipette, the measurement being repeated over time, the chart illustrating the need for a periodic repetition of pipette calibration, and the usefulness of the tracking of pipette status and renewal of calibration.

FIG. 2 shows the pipette calibration managing system (PCMS) 100 for managing the calibration of pipettes belonging to a pipette pool of a PCMS customer.

The PCMS 100 comprises a PCMS managing application 10 to be hosted by the PCMS customer and to be run by a PCMS host server 11, which is accessible for clients via a data network, indicated by a router or hub 5, to which the hardware components of the system as located on premise of the customer are connected, and which is connected to the internet via an internet router 6. The PCMS managing application 10 is configured for managing the access of the clients to PCMS data contained in a PCMS database 12 stored in a PCMS data storage 13 being located on premises of the PCMS customer.

The PCMS 100 comprises a client application 20 to be run by a client computer 21, in particular a mobile computer, for example a smart phone or a tablet PC, and configured to communicate with the PCMS managing application via the data network 5. The PCMS 100 comprises a further client application 30 to be run by a client computer 31 and configured to communicate with the PCMS managing application via the data network 5. The PCMS 100 comprises a further client application 40 to be run by a client computer 41 and configured to communicate with the PCMS managing application via the data network 5.

The PCMS 100 comprises a calibration ordering software 50 run by a calibration ordering computer 51, configured for
receiving a pipette ID number identifying a pipette of the pipette pool,
generating calibration order data containing a calibration order ID number and said pipette ID number,
providing the calibration order data to be transferred to a calibration service provider,

The PCMS managing application 10 is configured to receive calibration data from the calibration service provider via an external data network, in particular the Internet, as indicated by the connection via the internet router 6, and the PCMS database 12 is configured to store the calibration data, which thereby form at least a part of the PCMS data.

The calibration data contain the pipette ID number of said pipette and the calibration order ID number and the user ID number of the commissioning user. The calibration data contain calibration reporting data, which contain calibration status data and/or a calibration certificate, the calibration status data containing the information if a calibration of said pipette was successfully performed by the calibration service provider, or other status information, the calibration certificate containing the information certifying the authenticity of a successful calibration of the pipette.

Each client application 20, 30, 40 is configured to perform the following steps:
communicate with the PCMS managing application 10,
access the PCMS database 12,
receive the calibration reporting data in dependence on the pipette ID number and/or the calibration order ID number and/or a user ID number.

The calibration ordering software 50 is configured for
receiving a user ID number of a user belonging to the PCMS customer, the calibration order data containing also said user ID number,

and wherein the client application is configured to perform the following step:
receive the calibration reporting data in dependence on a user ID number.

The PCMS 100 comprises a calibration service managing software 60 to be hosted by the calibration service provider server 61 and configured to
receive the calibration order data from the calibration ordering computer 51 via a data network, in particular the Internet via router 6,
output the calibration order data for initiating the calibration of said pipette to be performed manually or automatically by the calibration service provider,
providing the calibration data,
transferring the calibration data to the PCMS database 12.

The PCMS 100 comprises, amongst others, the following hardware components:
the PCMS host server 11 and the PCMS data storage 13, both being located on premises of the PCMS customer,
the client computers 21, 31, 41, in particular being located on premises of the customer, and connected to the data network 5, and
the calibration ordering computer 51, in particular being located on premises of the customer, for running the calibration ordering software 50;
the calibration service provider server 61 being located on premises of the calibration service provider.

The PCMS 100 also comprises the following hardware component:
a calibration equipment 71, which is configured to perform the calibration. The calibration equipment includes a precision balance suitable, configured to measure the weight of volumes, which are relevant for the respective pipette to be calibrated, and a climate control chamber, which is capable to accommodate the precision weight and which is conditioned to standardized climate values, regarding a standardized temperature, and/or a standardized humidity, and/or a standardized gaseous composition of the surrounding air. The calibration is performed following ISO 8655 and ISO 17025.

The calibration equipment 71, in particular a calibration software 70 running on the calibration equipment, is configured to exchange data, in particular calibration data, with the PCMS 100, in particular with the PCMS managing application 10 and the PCMS database 12, wherein said data exchange is implemented to, by choice of the provider, be initiated automatically and substantially immediately after a calibration of a pipette is finished.

The PCMS 100, in particular the calibration ordering computer 51 and the calibration equipment 71, contain a reader 53, 73 for reading information from an information carrier, which is in particular attached to a pipette, and to enter data, which are derived from said information as read by the reader, into the PCMS, in particular for being used by a software of the PCMS.

The calibration service managing software 60 and the PCMS managing application 10 and the client application 20, 30, 40 are respectively configured to set, and output to the user, a renewal period of a pipette, or generally of all pipettes in the pool, the renewal period being the period between the date of a previously completed calibration and the date of a scheduled future calibration of a pipette.

The PCMS 100 also contains a reader 55 for reading the user badge of a user, which is in particular an RFID reader 55, which is connected to the calibration ordering computer 51.

FIG. 5 schematically shows a pipette calibration managing method (PCMM) 300 for managing the calibration of pipettes belonging to a pipette pool of a PCMS customer, the method 300 using, in particular, the pipette calibration managing system of FIG. 2 and comprises the steps:
receiving a user ID number of a user, who belongs to the PCMS customer, using the calibration ordering software, (301)
receiving a pipette ID number identifying a pipette of the pipette pool, using the calibration ordering software, (302)
generating calibration order data using the calibration ordering software, (303)
providing the calibration order data to be transferred to a calibration service provider, in particular via the Internet, (304)
receive and store calibration data from the calibration service provider via an external data network, in particular the Internet, in the PCMS database, the calibration data thereby forming at least a part of the PCMS data, (305)
let a user, via the client application: (306)
   access the PCMS database,
   receive the calibration reporting data in dependence on the pipette ID number and/or the calibration order ID number and/or the user ID number
   output the calibration reporting data to the user.

FIG. 6 shows a pipette 1, which may be part of the PCMS 100. The pipette 1 is an electronic pipette, having a computing device 7, which is connected with a data storage 4, forming an information carrier 4 for carrying data originating from the calibration, in particular calibration data, which were preferably automatically uploaded to the data storage 4 by the calibration equipment 71, immediately after the calibration of the pipette 1 was finished or within a predetermined time period after the calibration of the pipette 1 was finished. The information carrier may be part of the PCMS 100. A description of the technical function of 5 the information carrier is already contained in some of the previous paragraphs. The pipette 1 may contain a communication adapter, in particular using WLAN, for incorporating the pipette 1 into the data network (5, 6), which connects the hardware components of the PCMS 100. The pipette 1 has a display part 3 including a display 2, and a handling part 8, which contains an electric motor driving a pipetting piston inside the handling part 8 for applying a vacuum to an air cushion inside an air chamber of the pipette 1, thereby aspirating a liquid into the pipette tip 9.

1. A pipette calibration managing system (PCMS) for managing the calibration of pipettes belonging to a pipette pool of a PCMS customer, the pipette calibration managing system comprising:
a PCMS managing application to be hosted by the PCMS customer and to be run by a PCMS host server, which is accessible for clients via a data network and is configured for managing the access of the clients to PCMS data contained in a PCMS database stored in a PCMS data storage being located on a premises of the PCMS customer,
a client application to be run by a client computer and configured to communicate with the PCMS managing application via the data network,
a calibration ordering software configured for
receiving a pipette ID number identifying a pipette of the pipette pool,
generating calibration order data containing a calibration order ID number and said pipette ID number,
providing the calibration order data to be transferred to a calibration service provider,
the PCMS managing application being configured to receive calibration data from the calibration service provider via an external data network, in particular the Internet, and the PCMS database being configured to store the calibration data, which thereby form at least a part of the PCMS data,
the calibration data containing the pipette ID number of said pipette and/or
the calibration order ID number, and
the calibration data containing calibration reporting data, which contain calibration status data and/or a calibration certificate,
the calibration status data containing the information if a calibration of said pipette was successfully performed by the calibration service provider, the calibration certificate containing the information certifying the authenticity of a successful calibration of the pipette,
wherein the client application is configured to perform the following steps:
communicate with the PCMS managing application,
access the PCMS database,
receive the calibration reporting data in dependence on the pipette ID number and/or the calibration order ID number.

2. The pipette calibration managing system according to claim 1, wherein the calibration ordering software is configured for
receiving a user ID number of a user belonging to the PCMS customer, the calibration order data containing also said user ID number,
and wherein the client application is configured to perform the following step:
receive the calibration reporting data in dependence on a user ID number.

3. The pipette calibration managing system according to claim 1, which comprises:
a calibration service managing software to be hosted by a calibration service provider server and configured to
receive the calibration order data from the calibration ordering computer via a data network, in particular the Internet,
output the calibration order data for initiating the calibration of said pipette to be performed manually or automatically by the calibration service provider,
providing the calibration data,
transferring the calibration data to the PCMS database.

4. The pipette calibration managing system according to claim 1, which comprises one or more of the following hardware:
the PCMS host server and the PCMS data storage, both being located on the premises of the PCMS customer,
the client computer, in particular being located on the premises of the customer, and connected to the data network, and
the calibration ordering computer, in particular being located on the premises of the customer, for running the calibration ordering software;
and optionally: the calibration service provider server being located on a premises of the calibration service provider.

5. The pipette calibration managing system according to claim 1, which comprises one or more of the following hardware:
at least one calibration equipment, which is configured to at least contribute in performing the processes required for calibration or is configured to perform the calibration, and/or
at least one computer device configured to exchange data with the at least one calibration equipment and/or control the at least one calibration equipment.

6. The pipette calibration managing system according to claim 5, wherein the at least one calibration equipment includes
a precision balance configured to measure the weight of volumes, which are relevant for the respective pipette to be calibrated,
a climate control chamber, which is capable to accommodate the precision weight and/or which is conditioned to standardized climate values, regarding a standardized temperature, and/or a standardized humidity, and/or a standardized gaseous composition of the surrounding air.

7. The pipette calibration managing system according to claim 5, wherein the at least one calibration equipment 71), in particular a calibration software (70) running on the calibration equipment, is configured to exchange data, in particular calibration data, with the PCMS, in particular with the PCMS managing application and/or the PCMS database, wherein said data exchange is automatically and substantially immediately initiated after a calibration of a pipette is finished.

8. The pipette calibration managing system according to claim 1, wherein the PCMS, in particular the client computer, the calibration ordering computer, and/or the calibration service provider server, and/or the calibration equipment, contains a reader for reading information from an information carrier, which is in particular attached to a pipette, and to enter data, which are derived from said information as read by the reader, into the PCMS, in particular for being used by a software of the PCMS.

9. The pipette calibration managing system according to claim 1, wherein the calibration service managing software and/or the PCMS managing application and/or the client application is/are configured to set, and/or output to the user, a renewal period of a pipette, or generally of all pipettes in the pool, the renewal period being the period between the date of a previously completed calibration and the date of a scheduled future calibration of a pipette.

10. The pipette calibration managing system according to claim 9, wherein the calibration service managing software and/or the PCMS managing application and/or the client application may be configured to output in a display, or respectively in an output screen of a GUI, one or more or all of the following informations to the user:
an information about the renewal period of a pipette, or generally of all pipettes;
the ending date of the renewal period of a pipette, or generally of all pipettes, in particular regarding a specific user ID number
the date of the most recent calibration of a pipette, or generally of all pipettes, in particular regarding a specific user ID number, in particular more or all dated of calibration of said pipette.

11. The pipette calibration managing system according to claim 9, wherein the PCMS, in particular one of its software components, in particular the calibration service managing software and/or the PCMS managing application and/or the client application may be configured to evaluate and/or output to the user the renewal status of a pipette, which means that it is evaluated, and output, if needed, if a pipette, or respectively each pipette, at the current day of the evaluation:
first case (1; “normal phase”) lies within the renewal period of the respective pipette, and/or
second case (2; “reminder phase”) if the pipette, or respectively each pipette, is currently situated within a reminding period within the renewal period of the pipette, the reminding period ending with the end date of the renewal period. The reminding period may be set by a user, and may be 4 weeks by default, and/or
third case (3; “overdue phase”) is situated after the renewal period of the pipette.

12. The pipette calibration managing system according to claim 11, wherein the PCMS, in particular one of its software components, in particular the calibration service managing software and/or the PCMS managing application and/or the client application may be configured to graphically indicate the renewal status of one or more or all pipettes, in particular regarding a specific user ID number, to a user by outputting a graphical representation, in particular next to the pipette ID number of the one or more or all pipettes.

13. The pipette calibration managing system according to claim 12, wherein the graphical representation follows a color coding for the renewal status, for example applying the logic of traffic lights, wherein a first color, e.g. “green”, refers to the “normal phase”, a second color, e.g. “yellow”, refers to the reminder phase, and a third color, e.g. “red”, refers to the overdue phase of the renewal status.

14. The pipette calibration managing system according to claim 1, wherein the client computer is a handheld computer device, in particular a mobile phone or a tablet PC.

15. A pipette calibration managing method (PCMM) for managing the calibration of pipettes belonging to a pipette pool of a PCMS customer, the method using, in particular, the pipette calibration managing system according to claim 1 and comprising the steps:
optionally: receiving a user ID number of a user, who belongs to the PCMS customer, using the calibration ordering software,
receiving a pipette ID number identifying a pipette of the pipette pool, using the calibration ordering software,
generating calibration order data using the calibration ordering software,
providing the calibration order data to be transferred to a calibration service provider, in particular via the Internet,
receive and store calibration data from the calibration service provider via an external data network, in particular the Internet, in the PCMS database, the calibration data thereby forming at least a part of the PCMS data,
let a user, via the client application:
access the PCMS database,
receive the calibration reporting data in dependence on the pipette ID number and/or the calibration order ID number and/or the user ID number
optionally: output the calibration reporting data to the user.
